(19) United States
(12) Patent Application Publication
(10) Pub. No.: US 2003/0153546 A1
(43) Pub. Date: Aug. 14, 2003

(54) Microbicidal effect of prophyrines in darkness

(76) Inventors: Moshe Schaffer, Munchen (DE); Giulio Jori, Padova (IT); Alexander Holtz, Grunwald (DE)

Correspondence Address: Dale A Bjorkman, Kagan Binder, Suite 200 Maple Island Building, 221 Main Street North, Stillwater, MN, 55082, US

(21) Appl. No.: 10/169,274

(22) PCT Filed: Dec. 22, 2000
(86) PCT No.: PCT/DE00/04609

(30) Foreign Application Priority Data
Dec. 23, 1999 (DE) 199 62 505.0

(51) Int. Cl. A61K 31/444; C07D 487/22; A61K 31/555

(52) U.S. Cl. 514/185; 514/333; 540/145

(57) ABSTRACT

The invention relates to compounds of the formula:

| | |
|---|---|
| | (I) |
| 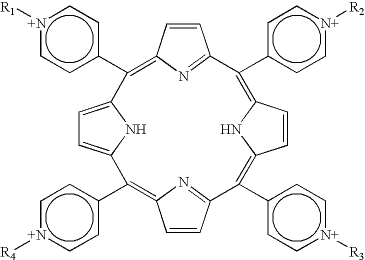 | |

| | | | |
|---|---|---|---|
| R₁ = R₂ = R₃ = | R₄ = | —CH₃ | T₄MPyP |
| —CH₃ | | —CH₂(CH₂)₄CH₃ | mono-C6 |
| | | —CH₂(CH₂)₈CH₃ | mono-C10 |
| | | —CH₂(CH₂)₁₂CH₃ | mono-C14 |
| | | —CH₂(CH₂)₁₆CH₃ | mono-C18 |
| | | —CH₂(CH₂)₂₀CH₃ | mono-C22 |
| | | 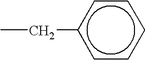 | mono-benzyl |
| R₁ = R₂ = R₃ = R₄ = | | | tetra-CS |
| —CH₂(CH₂)₄CH₃ | | | |

The present invention relates to porphyrine derivatives which have a microbicidal effect.

Porphyrines are photosensitizers, which absorb electromagnetic radiation, preferably visible light, and in this way catalyze the formation of radicals and/or singlet-oxygen from triplet-oxygen. Due to this radical mechanism living cells are partly or completely destroyed, and, thus, a possibility of controling harmful living organisms is presented. For example EP-A-0 390 743 discloses porphyrines and phthalocyanines as insecticides and acaticides. U.S. Pat. No. 4,648,992 discloses water-soluble phthalocyanine compounds, which can be used as bleaching agents or also for the disinfection of swimming pools. DEA-196 06 082 relates to a pesticide containing a photosensitizer of the tetrapyrrole and/or tetraazapyrrole series. In DE-A-196 06 081 similar tetrapyrrole photosensitizers are used to control bacteria in water.

In all of the above applications, however, the photosensitzers are only effective under the influence of electromagnetic radiation/light.

It has now been surprisingly found that certain porphyrine derivatives have a microbicidal effect even without the influence of electromagnetic radiation.

Consequently, according to the invention, compounds having the following general formula (I) are provided:

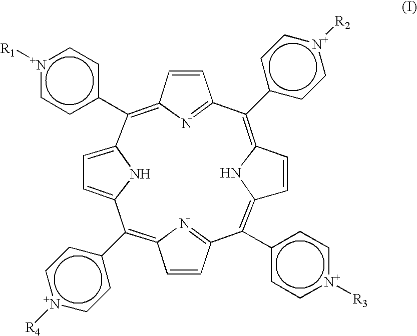

wherein R₁, R₂, R₃ and R4 are each independently optionally substituted alkyl or aralkyl groups. Preferably, the alkyl group is a C₁-C₃₀ alkyl group. Short alkyl groups having a chain length up to 4 C-atoms or longer groups having 6 to 22 C-atoms are particularly preferred. A preferred aralkyl group is the benzyl group. In one preferred embodiment, all groups R₁ to R₄ are short-chain alkyl groups having 1 to 4 C-atoms, particularly methyl groups. In one further preferred embodiment, three of the groups R₁ to R₄ are short-chain alkyl groups having 1 to 4 C-atoms, particularly methyl groups, and the fourth group is a longer, linear alkyl group having at least 6 C-atoms, a chain length of at least 10 C-atoms being more preferred. Compounds in which R₁, R₂, and R₃ are each a methyl group and R₄ is a C₆-C₂₂ alkyl group or a benzyl group, or a compound in which all groups R₁ to R4 are hexyl groups are also preferred. The microbicidal effect is enhanced by introducing one or more longer alkyl chains. The claimed compounds can be used as microbicidals, particularly bactericides.

The compounds according to the invention can be prepared in an analogous manner to already known porphyrine derivatives.

The effectivity of the inventive compounds will be explained in the following examples.

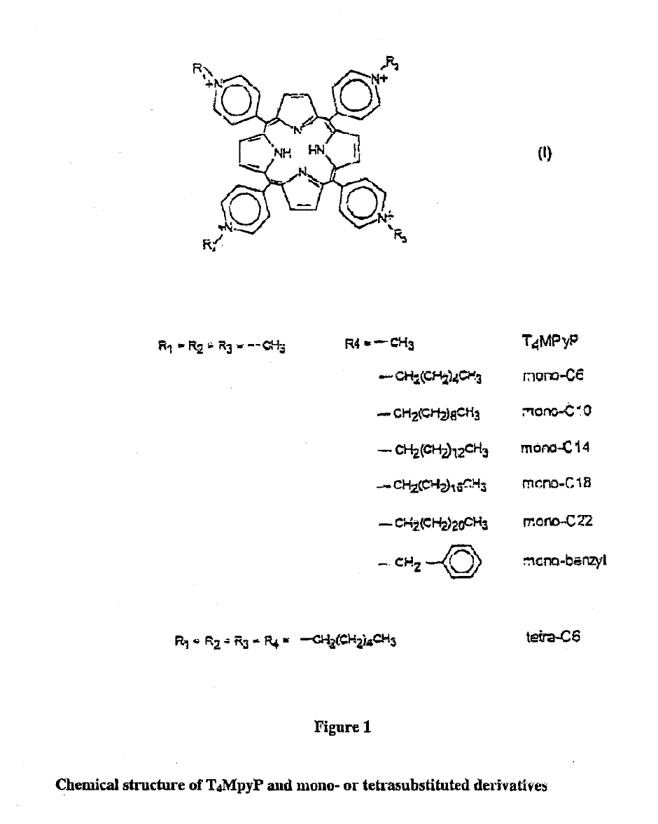

The definitions of the used porphyrine derivatives are to be taken from FIG. 1.

For this, porphyrines of different chemical structures (see drawing) were incubated with E. coli and S. aureus cells in the dark. The porphyrines were added to aqueous suspension comprising an amount of bacterial cells of 10⁸ per ml and stored for 5 minutes in the dark. Finally, the amount of porphyrine bonded to the cells was measured depending on the number of washing 6steps (the washing was carried out by means of physiological solutions) by chemical extraction followed spectrophotofluorimetric determination.

The survival rate of the bacterial cells after incubation with the different porphyrines was determined by diluting 0.3 ml of the suspension with 2.7 ml of the culture medium and recording the optical density at 650 nm depending on the time. The suspensions were kept at 37° C. The percentual inhibition of growth was calculated with reference to a suspension of comparative cells which had been treated with the same method, however had not been added to the porphyrine.

TABLE 1
Recovery uf porphyrine flow E. coli cells which have been incubated
with 1 μM of porphyrine for 5 minutes and subjected to different
washing steps

| | | | | % |
|---|---|---|---|---|
| | recovery (nmol/mg protein) | | | bonded |
| | | 1 washing | 3 washing | after |
| porphyrine | no washing | step | steps | 3 washing steps |
| T₄MPyP | 0.44 ± 0.04 | 0.19 ± 0.04 | 0.05 ± 0.01 | 11.4 |
| mono-C6 | 1.58 ± 0.28 | 1.01 ± 0.12 | 0.39 ± 0.12 | 24.7 |
| mono-C10 | 3.66 ± 0.071 | 2.78 ± 0.53 | 1.48 ± 0.38 | 40.4 |
| mono-C14 | 5.38 ± 0.06 | 3.06 ± 0.25 | 2.28 ± 0.04 | 42.4 |
| mono-C18 | 4.20 ± 0.16 | 4.08 ± 0.62 | 2.54 ± 0.38 | 60.5 |
| mono-C22 | 5.12 ± 0.46 | 2.60 ± 0.04 | 3.04 ± 0.34 | 59.4 |
| mono- | 2.52 ± 0.13 | 1.67 ± 0.06 | 0.71 ± 0.08 | 28.2 |
| benzyl | | | | |
| tetra-C6 | 1.65 ± 0.17 | 1.39 ± 0.23 | 0.94 ± 0.24 | 57.0 |

TABLE 2
Recovery of porphyrine from S. aureus cells which have been
incubated with 1 μM of porphyrine for 5 minutes in the dark and
subjected to different washing steps

| | | | | % |
|---|---|---|---|---|
| | recovery (nmol/mg protein) | | | bonded |
| | | 1 washing | 3 washing | after |
| porphyrine | no washing | step | steps | 3 washiug steps |
| T₄MPyP | 0.68 ± 0.05 | 0.10 ± 0.01 | 0.11 ± 0.01 | 16.2 |
| mono-C6 | 3.04 ± 0.05 | 2.05 ± 0.11 | 1.02 ± 0.12 | 33.6 |
| mono-C10 | 7.13 ± 0.25 | 5.70 ± 0.54 | 4.09 ± 0.13 | 57.4 |
| mono-C14 | 6.56 ± 0.40 | 5.53 ± 0.74 | 4.32 ± 0.13 | 65.9 |
| mono-C22 | 6.39 ± 0.40 | 5.65 ± 0.13 | 4.95 ± 0.30 | 77.5 |

TABLE 3
Effect of the 5-minute incubation in the dark of E. coli and S. aureus
cells with the different porphyrines on the survival rate of the
bacterial cells

| | concentration | inhibition of growth (%) | |
|---|---|---|---|
| porphyrine | (μM) | E. coli | S. aureus |
| T₄MPyP | 1 | 0.6 | 3.1 |
| | 10 | 3.1 | 7.8 |
| mono-C6 | 1 | 0.5 | 5.1 |
| | 10 | 14.6 | 8.8 |
| mono-C10 | 1 | 4.6 | 27.8 |
| | 10 | 10.7 | 50 |
| mono-C14 | 1 | 46.1 | 69.1 |
| | 10 | 100 | 92.4 |
| mono-C18 | 1 | 2.7 | 39.8 |
| | 10 | 85.7 | 96.6 |
| mono-C22 | 1 | 19.1 | 39.9 |
| | 10 | 16.8 | 70.1 |
| mono-benzyl | 1 | 0.1 | 1.6 |
| | 10 | 13 | 25 |

1. A compound of the following general formula: 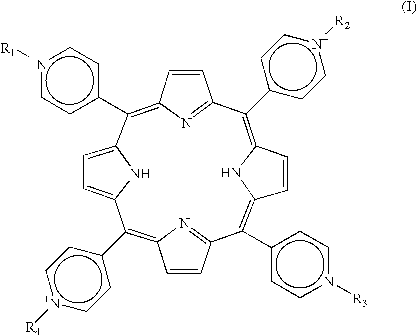
wherein R₁, R₂, R₃ and R₄ are each independently optionally substituted alkyl or aralkyl groups.

2. The compound according to claim 1, wherein R₁, R₂, R₃ and R₄ are all methyl groups.

3. The compound according to claim 1, wherein R₁, R₂, R₃ and R₄ are each hexyl groups.

4. The compound according to claim 1, wherein R₁, R₂ and R₃ are each a methyl group and R₄ is a straight-chain C₆, C₁₀, C₁₄, C₁₈, C₂₂ alkyl group or a benzyl group.

5. Use of the compound according to claim 1 as a microbicide.

6. Use according to claim 5, essentially effected in the absence of visible and UV-light (in the dark).

7. Use according to claim 5, essentially effected alternately in the dark as described in claim 6 and under the influence of visible and UV-light.

8. Use according to claim 7, the wavelength of visible and UV-light being in the range of 350 to 900 nm.
